(19) United States
(12) Patent Application Publication
(10) Pub. No.: US 2015/0112170 A1
(43) Pub. Date: Apr. 23, 2015

(54) DEVICE AND METHOD FOR NON-INVASIVE GLUCOSE MONITORING

(71) Applicant: Amerson, LLC, Los Angeles, CA (US)

(72) Inventors: Robert Lee Amerson, III, Los Angeles, CA (US); Robert Lee Amerson, II, Los Angeles, CA (US); Elizabeth Roshae Amerson, Los Angeles, CA (US)

(73) Assignee: AMERSON, LLC, Los Angeles, CA (US)

(21) Appl. No.: 14/517,778

(22) Filed: Oct. 17, 2014

Related U.S. Application Data
(60) Provisional application No. 61/891,903, filed on Oct. 17, 2013.

(51) Int. Cl. A61B 5/1455; A61B 5/00; A61B 5/145

(52) U.S. Cl. CPC A61B 5/1455; A61B 5/14532; A61B 5/742; A61B 5/746; A61B 5/7207; A61B 5/681
USPC 600/316; 600/310; 600/322

(57) ABSTRACT

A device and method for non-invasively measuring analytes and physiological parameters by measuring terahertz radiation emitted though biological tissue. Terahertz pulses are emitted from a miniaturized quantum cascade laser to a fiber optic array into the wrist of the user. A corresponding sensor on the opposite side of the wrist receives the terahertz signals that have been modified by interacting with organic molecules. The data from the sensor is compiled and analyzed on a RAM chip and logic chip, where a program uses an algorithm to compare measurements to a library of existing measurements and topographic maps generated when the user first dons the device. Once the algorithm has parsed all the data points, a value, such as blood glucose level, appears on a display of the device. The device may be equipped with a gasket to reduce ambient light from contacting the sensor.

### CROSS-REFERENCE TO RELATED APPLICATIONS

This patent application claims the benefit of U.S. Provisional Patent Application No. 61/891,903, filed on Oct. 17, 2013, hereby incorporated by reference in its entirety.

### FIELD OF THE DISCLOSURE

The present invention relates to a non-invasive device and method for measuring analytes and physiological parameters in a biological being. More particularly, the device and method measure glucose concentration by sensing the absorption of terahertz waves emitted through human tissue.

### BACKGROUND OF THE INVENTION

Diabetes (type I and II) is potentially life threatening, but with studious management, a person living with the disease can live a full, normal and active life. However, current technologies for the daily monitoring of the disease are often cumbersome, painful and invasive. The standard procedure is for the diabetic person to break his or her skin, draw blood, capture blood on a strip, and insert the strip into a measuring device. Not only is measuring glucose in this way painful, but it is also difficult and expensive to perform constant monitoring of one's own blood glucose level.

There have been several attempts to monitor glucose levels non-invasively using various methods and devices, but these attempts have been met with challenges and have not been proven to be very successful. Some current non-invasive devices require direct contact with skin, use electric current, and/or use adhesives. These devices and methods often produce a skin irritating effect and are inaccurate. Other ways to measure blood glucose non-invasively have included: shining light through skin or body tissues, using ultrasound, blood viscosity testing, and measuring infrared radiation emitted by the body. Some technologies measure glucose and other analytes by measuring reflection and/or of absorption of electromagnetic waves in the terahertz range (approximately 0.3 to 3.0 terahertz). By measuring reflection or absorption of terahertz radiation by organic molecules, and comparing these measurements with a database of known reflection/absorption values for concentrations of organic molecules can be determined. Some technology used to measure blood glucose levels are disclosed in the following patents and patent applications:

U.S. Pat. No. 6,188,648 to Olsen discloses a diabetic care watch that signals the wearer of the watch for a need to test blood glucose levels. Here, the wearer manually calculates carbohydrates counts and blood glucose levels are not directly measured.

U.S. Pat. No. 7,174,199 to Berner discloses a method and device for measuring blood glucose levels transdermally using iontophoresis.

U.S. Pat. No. 8,135,450 to Esenaliev discloses a non-invasive method and system to detect blood glucose levels based on the change of tissue dimensions, which correlate to blood glucose concentration.

U.S. Pat. No. 8,698,085 to Ouchi discloses an apparatus to measure analytes in a gas (not within human tissue) using terahertz or infrared radiation.

U.S. Patent Application Pub. No. 20070255122 to Vol discloses a device to measure analytes. The device uses at least two spaced apart electrodes for providing a bio-potential measurement to determine physiological parameters.

U.S. Pat. No. 6,675,030 to Ciurczak discloses an individualized modeling equation for predicting a patient's blood glucose level generated as a function of non-invasive spectral scans of a body part and an analysis of blood samples from the patient.

U.S. Pat. No. 6,645,142 to Braig discloses a glucose monitoring instrument having network-based communication features that provide a link between patient and practitioner.

U.S. Pat. No. 6,723,048 to Fuller discloses an apparatus for non-invasive detection and quantification of analytes, such as blood glucose, by employing an amplifier that uses high-gauss permanent magnets to permit an RF signal to be transmitted through a sample. The concentration of the analyte can be determined from the magnitude of the reduction in the amplitude of the radio-frequency (RF) signal at a characteristic frequency.

U.S. Patent Application Pub. No. 20080068932 to Mosley discloses a watch for monitoring diabetes, which includes an alert system, and includes measurement by a transdermal sensor mounted to the back of a wristwatch.

U.S. Pat. No. 6,923,763 to Kovatchev discloses a non-linear model and implementation for hypoglycemia that uses predictive algorithms for determining the onset of hypoglycemia.

U.S. Patent Application Pub. No. 20130289370 to Sun discloses a method and device using an electromagnetic absorption constant.

International Patent Application Pub. No. WO2007071092 to Artley discloses a non-invasive blood glucose sensor that uses terahertz radiation to detect blood glucose levels by measuring reflected radiation.

Patents and patent applications that teach hardware and software implementation are generally known, and are disclosed in U.S. Pat. No. 4,858,207 to Buchner, U.S. Pat. No. 5,371,687 to Holmes, U.S. Pat. No. 5,678,571 to Brown, and U.S. Pat. No. 5,701,894 to Cherry.

Despite the advances in non-invasive glucose monitoring, all suffer from one or more drawbacks in accuracy, comfort, convenience, features, and price. Therefore, there is a continuing need for new devices and methods that accurately and non-invasively measure physiological parameters and analytes, such as blood glucose.

### BRIEF SUMMARY OF THE PRESENT INVENTION

The present invention provides a device and method for measuring analytes and physiological parameters in a biological being. Analytes in blood that are desirous of measuring, include, but are not limited to: glucose, urea, lactate amino acids, enzyme substrates, and products indicating a disease state or condition.

In one aspect of the present invention, the invention provides a device that allows an individual, parent, guardian, or medical professional, a consistent, non-invasive way to continuously, or nearly continuously monitor blood glucose levels in children or dependent elders with type I or type II diabetes. The device may be in the form a wristband or wristwatch, which enables a user to be alerted to dangerously high or low levels of glucose. These and other objects are accomplished using a combination of the following: (a) a miniaturized quantum cascade laser (QCL) designed to emit a plurality of pulses of terahertz radiation that are tuned to the resonant frequency of the analyte (e.g. glucose) being sampled, (b) an emitter unit operatively connected to the miniaturized quantum cascade laser where emitter unit emits the terahertz radiation from a fiber optic emitter array that has an array of field emission points arranged two-dimensionally on the fiber optic emitter array, (c) a sensor unit, preferably comprising a photo-conductive indium antimonide sensor array that is adapted to detect terahertz radiation generated by the QCL, (d) a display unit adapted to display at least one measurement of an analyte measured by the device, and (e) a processing unit (such as a CPU) that has a stored programmable memory and a random access memory. The processing unit is configured to process signals received by the sensor, and determine the concentration of analytes, or measure other physiological parameters. The processing unit may be connected to programmable memory, a random access memory, the QCL, the sensor unit, and display unit. Analytes such as glucose may be specifically measured when the terahertz frequency is tuned to a frequency that excites specific analytes, and produces a unique optical excitation spectra that can be analyzed. The processing unit compares readings from the sensor to an onboard database of similar body types and compositions, and uses an algorithm to determine the concentration of the analyte by comparing data from a user to database of optical spectra of various analytes, similar body types, and compositions. The measurements may be shown to the user on a display (such as the face of a wristwatch), and may also be transmitted to a third party.

In one aspect of the device, the miniaturized quantum cascade laser is operatively connected through fiber optic array that has approximately 350 terahertz fiber optic emission points. The fiber optic array is aligned to a corresponding indium antimonide sensor on the opposite side of the user's tissue (e.g. wrist).

In another aspect of the device, the fiber optic sensor is surround by a comfortable opaque material (such as a neoprene gasket, which may have inflatable features) to reduce ambient light form contacting the sensor, and provides a secure fit.

In another aspect of the device, the device includes a universal serial bus (USB) connector so that data from the device can be retrieved and the device can be charged via the same connector.

In another aspect of the device, the device includes a wireless transmitter capable of transmitting data to third party.

In another aspect of the invention, the present invention provides a non-invasive method for detecting the concentration of an analyte or physiological parameter. The method includes steps of: (a) generating electromagnetic waves in a terahertz range using a device comprising a miniaturized quantum cascade laser (QCL), (b) emitting electromagnetic waves in a terahertz range via a fiber optic array having plurality of field emission points arranged two-dimensionally, (c) transmitting electromagnetic waves in the terahertz range through a biological tissue, (d) measuring transmitted electromagnetic waves on a photo-conductive sensor array, wherein the photo-conductive sensor array comprises a plurality of individual photo-conductive sensors arranged two-dimensionally, and wherein the photo-conductive sensor array is positionally arranged parallel to the fiber optic array on opposite sides of the biological tissue, and (e) calculating a value of an analyte from the transmitted waves by determining a frequency energy received by the photo-conductive sensor having a plurality of photo-conductive sensors.

In other aspects of the invention, the method includes emitting the electromagnetic waves only when the device is relatively flat and still, and the sensor is relative dark, in order to prevent erroneous due to momentum, motion, and shifting of internal tissues, and ambient light that that may lead to compromised results.

In yet another aspect of the method, the method includes a step of calibrating the device to account for discrepancies that may occur due to non-blood tissues near the emitter (e.g., bone, tendons, etc.).

### BRIEF DESCRIPTION OF THE DRAWINGS

FIG. 1a is a perspective view of a wristband used to house a glucose detection device.

FIG. 1b is a top cross sectional view of the wristband of FIG. 1a.

FIG. 2a is a perspective view of an engine and housing for a glucose detection emitter array.

FIG. 2b is the bottom view of engine and emitter array of FIG. 2b.

FIG. 3 is an exemplary view of a cascade quantum laser and array embedded within the wristband.

FIG. 4 is an exemplary view of sensor array embedded within a wristband that shows an individual photo-conductive sensor within the array.

FIG. 5 is cross sectional exemplary view of the glucose detection device within a wristband around a user's wrist.

FIG. 6 is a cross sectional view of a wristband having a gasket to block ambient light from contacting the sensor.

FIG. 7 is a system diagram of the glucose monitoring device.

### DETAILED DESCRIPTION OF EMBODIMENTS

The following discussion addresses a number of embodiments and applications of the present disclosure. Reference is made to the accompanying drawings that form a part hereof, and are shown by way of illustration of specific embodiments in which the disclosure may be practiced. It is to be understood that other embodiments may be utilized and changes may be made without departing from the scope of the present disclosure. It is to be understood that the present disclosure is not limited to such specific application and that numerous implementations of the present disclosure may be realized.

The beneficial features of the present disclosure will be evident from the described embodiments. All references to patents, patent applications, and non-patent publications in the background and description are hereby incorporated by reference, in their entireties.

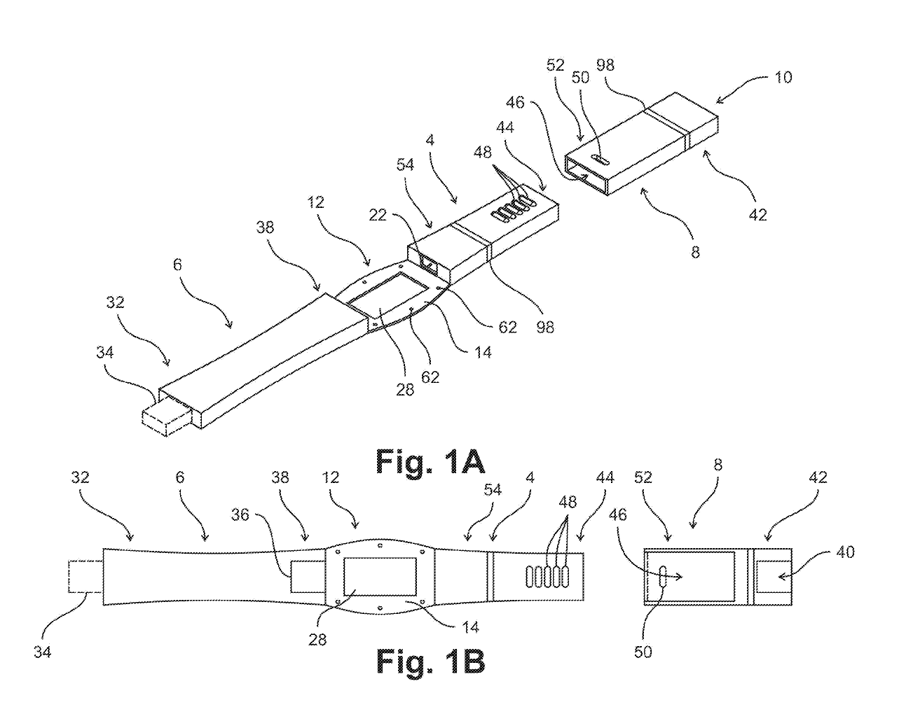

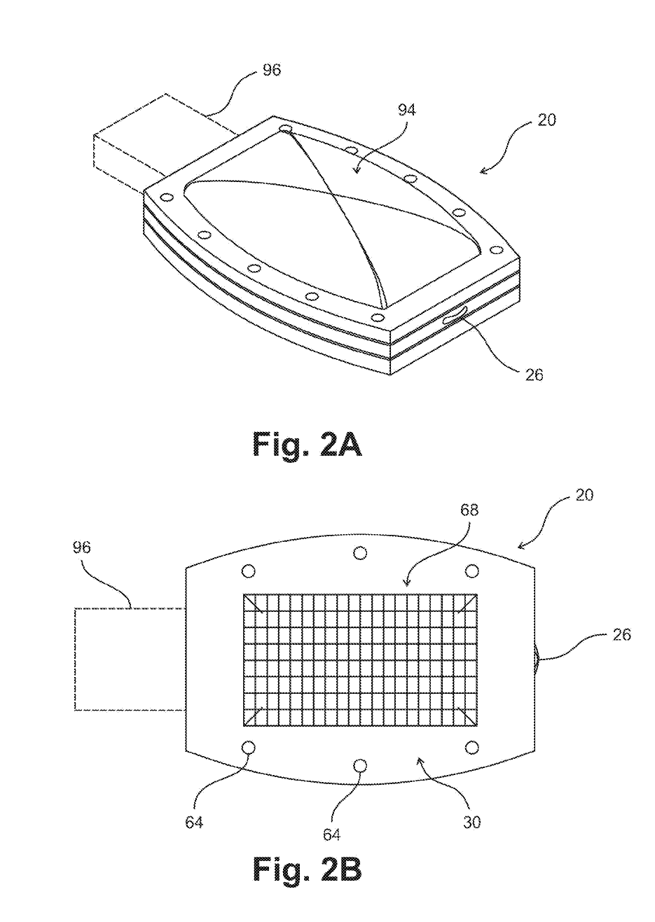

In one embodiment of the invention, a glucose detection device is in the form of a wristband 10 and more specifically a wristwatch, as shown in FIG. 1a and FIG. 1b. Other embodiments may include the technology incorporated in a ring, earring, or other wearable device. The wristband 10 includes an upper strap 4, a lower strap 6, and adjustable strap 8. Connecting upper strap 4 and lower strap 6 is a pocket 12 that has a top surface 14 and a bottom surface (not shown). The engine 20 (See FIGS. 2a and 2b) of the device is placed within the pocket 12. Defined here, the engine 20 comprises the internal software and some hardware of the device, such as the sensor unit 84 and display 94. The engine 20 may operate independently of the band so that the user can remove the engine 20 from the wristband 10 if he or she desires. The engine 20 may be worn once fully assembled into the wristband 10. The engine 20 is small enough so that it may discreetly fit inside a purse, wallet, pocket, or keychain, where the user may manually check blood sugar levels whenever he or she pleases. The engine 20 may be made out of a variety of materials such as anodize aluminum, or metallic microlattice, and may have a glass curved touchscreen 94. The engine 20 may securely connect to the band via a variety of means, and is illustrated as a hook 22, or other type of protrusion near the first end 54 of the upper strap 4. The protrusion 22 is designed to latch onto a fastening, or shackle 26 of the engine 20. The hook may be made from any sturdy material, but preferably steel. The bottom surface of the pocket 12 has a hole 28 so that when the engine 20 is placed in the pocket 12, the bottom surface of the engine 20 is placed in direct contact with the user's skin, or may be slightly raised from the user's skin. The engine 20 may also have a plurality of protrusions 64 that align with indentations 62 located on the top surface 14 of the pocket in order to help lock in the position of the engine 20 within the pocket 12.

The lower strap of the watch 6 has a USB port (male) 34 at a first end 32, and a USB port (female) 36 at its second end 38. The adjustable strap 8 has a USB port (female) 40 at its first end 42 and insertion hole 46 to insert the second end 44 of the upper strap 4. A plurality of adjustment protrusions 48 along the length of the top surface of the upper strap 4 corresponds in size and shape to an adjustment hole 50 on the second end 52 of the adjustment strap 8. The wristband 10 can therefore vary in length depending on which protrusion 48 is secured to the adjustment hole 50. To secure the entire wristband 10 around the user's wrist, the USB port (male) 34 of the lower strap 6 is inserted into the USB port (female) 40 of the adjustment strap 8. The upper strap 4, lower strap 6, and adjustable strap 8 may include various ornamental features such as aluminum bands 98 along one or more regions of the straps.

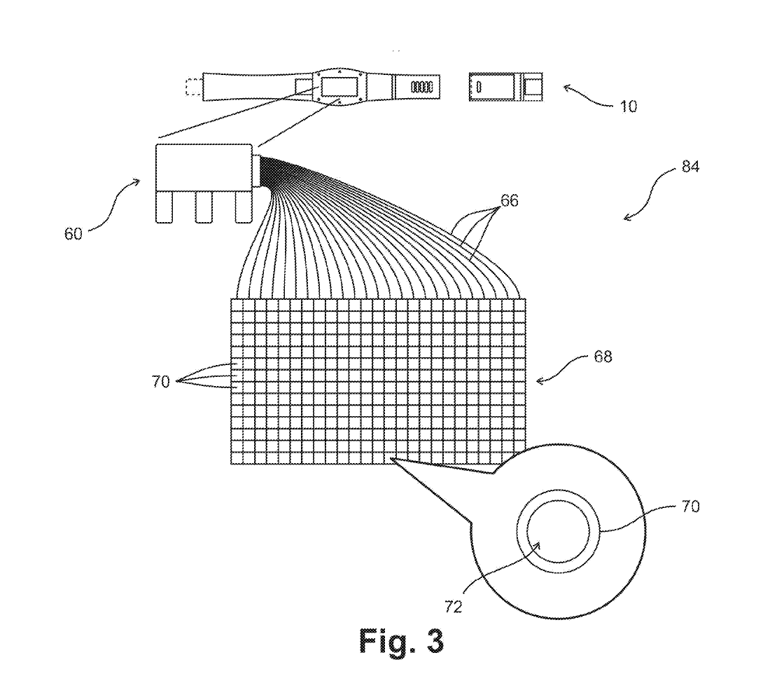

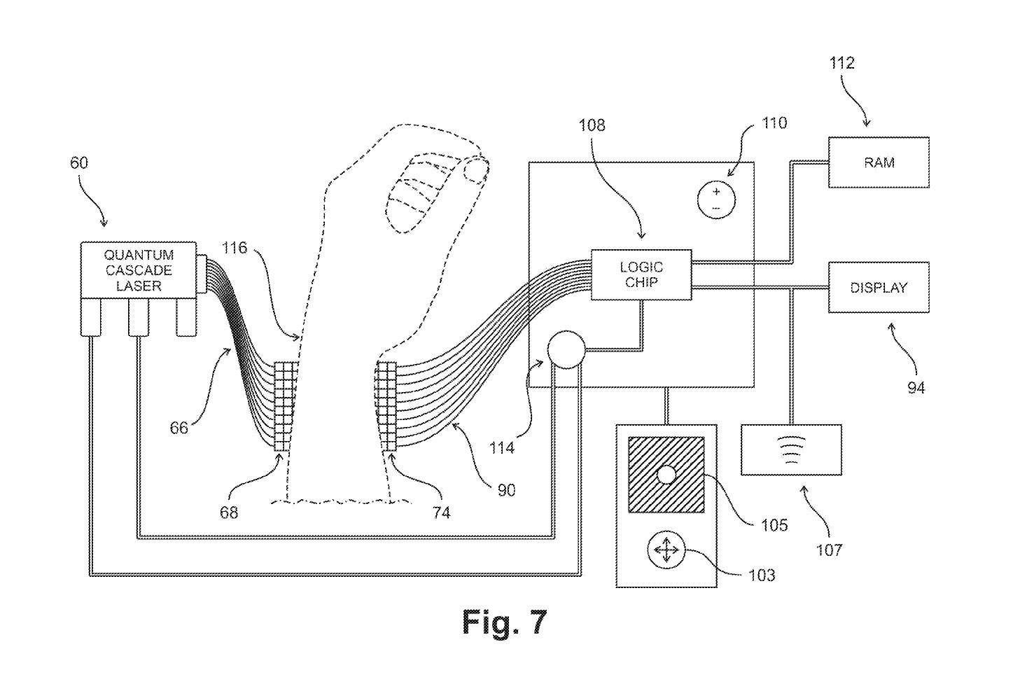

Referring now to FIGS. 2a and 2b, the engine 20 includes a miniaturized quantum cascade laser (QCL) 60 capable of emitting radiation of terahertz frequency (shown in FIG. 3). The use of QCLs for emitting terahertz radiation is known in the art. (See “Continuous Glucose Monitoring by Means of Fiber-Based, Mid-Infrared Laser Spectroscopy,” Labrecht, et al., Applied Spectroscopy 60, 729-736 (2006)). Terahertz radiation has also been used to detect a variety of polycrystalline structures. (See “Far-infrared Vibrational Modes of Polycrystalline Saccharides” Upadhya, et al., Vibrational Spectroscopy, 35, 129-143 (2004)). In the present embodiment, the engine 20 has an emitting unit 84 that is connected to the miniaturized QCL 60. The emitting unit 84 may have a tuning module 114 connected to a controller or processor 108 that allows the wavelength of the terahertz radiation emitted by the laser 60 to be controlled within a range of frequencies. The operative connections between various components of the device are illustrated in FIG. 7. By default, the device will be set to detect glucose at or around 1.4 terahertz (or a close range, such as between 1.3 and 1.5 terahertz). The laser 60 can be tuned to emit and detect other substances if desired. For example, to detect fructose, the laser 60 can be tuned to approximately 1.7 terahertz, well within the QCL's operating range.

In operation, the beam of the laser is guided through the terahertz equivalent of fiber optic strands 66 and the terahertz waves are emitted at the end of each strand on fiber optic emission points 72, which are mounted to an array 68 or grid of individual emitting cells 70. The array 68 may be in the form of a mounting lattice made of a flexible, insulating material. The lattice may be comprised of a nylon-polycarbonate material, such as those manufactured by Taulman 3D, LLC. Array lattices made from a nylon-polycarbonate hybrid have advantages in that they exhibit both strong insulating and flexion qualities and they may be manufactured additively (i.e. 3D printing). The array 68 may be of a variety of sizes and shapes. In a preferred embodiment, the emitter array 68 is a size that comfortably lies substantially flat on the top of a user's wrist 116 or other body part. In a preferred embodiment the array 68 is approximately 0.57 inches (1.45 cm) in length, and 1.00 inches (2.54 cm) in width. The size of the array 68 may be doubled (1.04 in. by 2 inches) or halved (0.29 in. by 0.50 in.) without detracting from the comfort or utility of the device. However, any size that would fit comfortably on the user's wrist would work equally well. Preferably, the size of each emitting array cell 70 on the array 68 is approximately 1 mm×1 mm, but sizes that are double of half of the preferred size would likewise not detract from the comfort or utility of the device. Each cell 70 of the array is operatively connected to a fiber optic strand 66. An exploded view of a single emitting cell 70 is shown in FIG. 3 as well as a sample of numerous fiber optic strands 66 attached to the array 68.

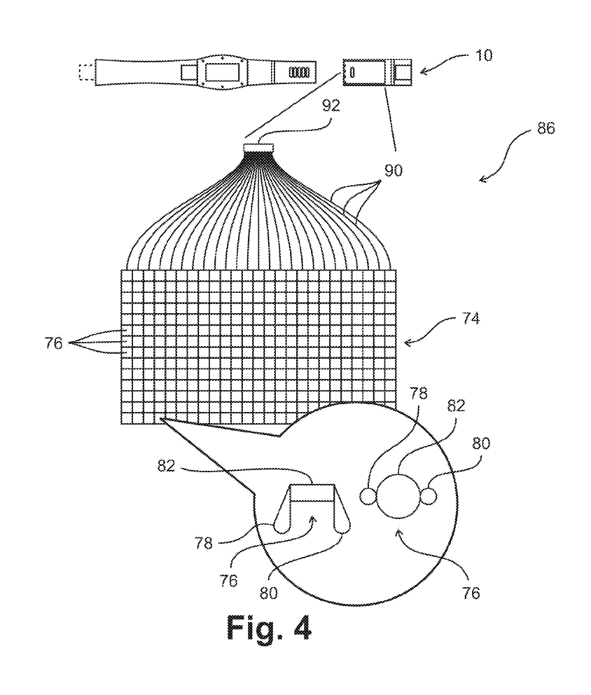

Within wristband 10 is a sensing unit 86 that connects a sensor array 74 via serial cables 90 to a voltage detecting unit 92 (shown in FIG. 4). The sensor array 74 has the same dimensions and same number and size of cells as the emitter array 68. The individual photo-conductive sensors 76 on the array 74 are arranged two-dimensionally, and each comprises a positive terminal 78, a negative terminal 80, and a disc 82 of photo-conductive indium antimonide mounted between the positive and negative terminals 78, 80. Depending on the frequency of the terahertz radiation received, more or less current may pass through each sensor. Other photoconductive materials may be used in the sensor, such as: indium arsenide, mercury telluride, cadmium mercury telluride, lead telluride, gallium arsenide, aluminum arsenide, aluminum nitride, aluminum phosphide, boron nitride, boron phosphide, boron arsenide, gallium antimonide, gallium nitride, gallium phosphide, indium phosphide, cadmium zinc telluride, and alloys and/or mixes of the above. Indium antimonide sensors and sensors other of semi-conductive sensor arrays are known in the art and disclosed in U.S. Pat. No. 7,026,602 to Dausch, U.S. Pat. No. 5,580,795 to Schimert, U.S. Pat. No. 8,324,660 to Lochtefeld, U.S. Pat. No. 7,864,326 to Cox, and U.S. Pat. No. 8,809,106 to Cheng.

The sensor unit 86 may be embedded in the strap so that the sensing array 74 lies directly on the skin of the user. In a preferred embodiment, in order to reduce interference on the array from dead skin cells, dirt, sweat, or other matter, the sensor array 74 may be slightly recessed from the surface of upper wristband 10 so that the sensor array 74 in not direct contact with the user's skin when the wristband 10 is wrapped around the user's wrist 116.

Since the glucose measurement device is wearable, this enables a user to be immediately alerted to dangerously high or low levels of glucose. Alerts may be accomplished using a variety of methods. The alert may be an audible alarm on the device that signals the user to dangerously high or low glucose levels. The glucose measurements may be shown to the user on a display 94 (such as the face of a wristwatch) and may also be transmitted to a third party, wirelessly via blue-tooth, RF, 3G, 4G, Wi-Fi other known wireless technology.

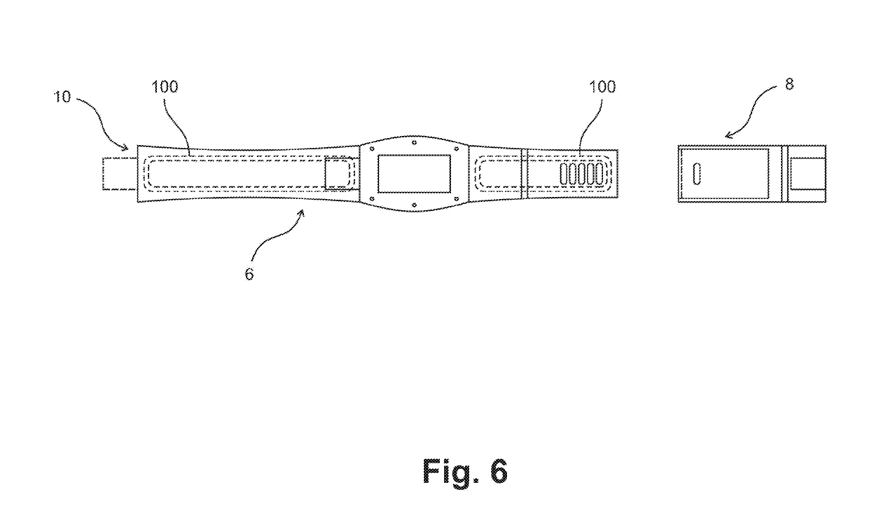

In a preferred embodiment there are features of the device that prevent outside ambient light from exciting the sensor array 74, which would cause the sensor's photoconductivity to spike, essentially blotting out the desired reading from the sensor array 74. A set of darkening tracks, which may be in the form of a gasket 100, trace the edges of the wristband 10, as illustrated in FIG. 6. The gasket 100 may be made from a microfiber wrapped around a core of neoprene or similar material that has a hollow air channel in its center, which allows air to inflate within the gasket 100 and the air pressure is used to tighten or loosen the wristband 10 against the user's wrist. The gasket 100 is connected to piping that is connected to a small pump that pumps air into the gasket 100. Air may be pumped into the gasket material itself, or in some embodiments the gasket 100 is in the form of a closed hollow rectangular, circular, or doughnut shape where air can be pumped inside of the hollow region. The gasket 100 prevents air from leaking out from the edges of the wristband 100 due to the airtight features of the gasket material and wristband 10 material against the user's skin. In another embodiment, water may be pumped in the piping instead of air, which has the added benefit of eliminating interference from any environmental terahertz radiation. The gasket feature is preferable to traditional clinching because of the need for symmetrical positioning of the emitter array and the sensor array. Inflatable technologies have been used in other devices to measure pulse, such as the pump band disclosed in U.S. Pat. No. 5,509,423 to Bryars and physiological sensing device in U.S. Pat. No. 6,491,647 to Bridger. Before the measuring device takes a glucose reading, ambient light should be below a threshold so that the essential reading is not blotted out by the presence of excessive ambient light.

The power to pump air into the gasket 100, and power the device in general, may be through an integrated cable through which the unit may be charged and also charge a pair of miniaturized air pumps. By pumping air into the gasket, not only is ambient light preventing from contacting the sensor 74, but the emitter 68 and sensor 74 are stabilized into a more fixed position on the user's wrist, which provides for more accurate readings.

The device as a whole is preferably powered by a lithium ion battery 110 or similar battery, which can be recharged via the built-in USB connection 96 in the same manner as a smart phone. The device preferably may be charged while fully assembled, and may also be charged by connecting the device to a wall outlet. The engine 20 of the device may also connect to a charger separately.

The display 94 of the engine 20 can be a LED or LCD display similarly found on any digital watch. The display 94 would also have a three-digit area to display blood glucose levels. Other areas could display measurements of other physiological parameters (such as other analytes, body temperature, blood pressure, etc.). The display 94 may have ancillary functions as desired, and may be a touchscreen display controlled by an operating system. The screen can also serve as an interface for calibrating the watch, setting up readings, reporting frequencies, and tuning the QCL to detect other analytes besides glucose. Multiple wireless communication options (Bluetooth, Wi-Fi, 3G, 4G, 5G, LTE, RF radio, etc.) could be embedded within device to wirelessly transmit the recorded physiological parameters from the device to another user via a transmitting module 107. The device may be equipped with components so that data can be sent by SMS/MMS, send information to a calendar/messenger, or send alerts/conventional alarms. As the final readings take very little storage space, the device can potential archive years of data onboard, however, a back up is preferred.

The device may have an embedded system on a chip (SoC) 108 such as an A8 chip, or similar chip known in the art. It may include a microcontroller, a microprocessor, a DSP core, memory blocks (ROM, RAM 112, flash memory), and interfaces for USB, Firewire, or Ethernet. Preferably, there is at least 2 GB of RAM. Preferably there is a separate module to tune the QCL's frequency tuning mechanisms such as those disclosed by Lu et al. in “Widely-tuned room-temperature terahertz quantum cascade laser sources” SPIE Proceedings, Vol. 8631, p. 863108-1, Photonics West, San Francisco, Calif., by Lu et al. (Feb. 3, 2013), and “Widely tuned room temperature terahertz quantum cascade laser sources based on difference-frequency generation” in Applied Physics Letters, Vol. 101, No. 25, p. 251121-1 (Dec. 17, 2012).

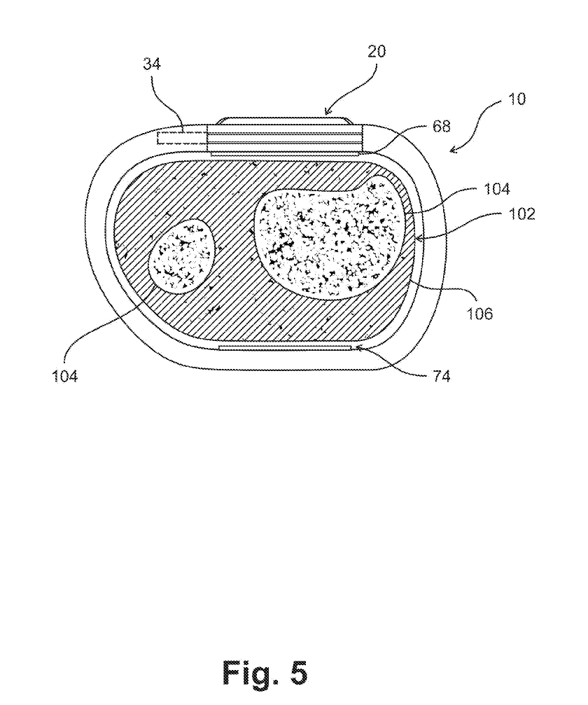

FIG. 5 illustrates a cross-sectional view of the device on a user's wrist. The wrist comprises various bones 104, and soft tissue 106, which may includes blood, adipose, and adipose. The emitting array 68 is aligned with the sensing array 74 on the opposite side of the user's wrist 102. When the arrays 68, 74 are aligned, the device can detect the amount of glucose or other analyte by detecting the absorption of terahertz radiation at specific frequencies emitted from the emitting array 68 and detected by the sensor 74. In another embodiment, instead of a single emitting array 68 and sensor array 70, the device may have several emitting and sensor arrays interspersed along the wristband 10. This may have the added benefit of obtaining more readings and it would not limit the terahertz beams from necessarily having to traverse the entirety of the wrist, but instead the sensor may measure reflected excitation of terahertz radiation from organic molecules, instead of measuring transmitted radiation. In some embodiments the sensor cells may be arranged in a pattern of tessellated hexagons approximately 1 mm in diameter with terahertz fiber optic emission points threaded trough vertices. This would allow the individual sensors 76 to measure the interactions of the terahertz radiation at the sub-dermal layer without the need to transverse the full thickness of the wrist.

In order to prevent compromised readings the device may be equipped with one or more than one of an accelerometer 103, gyroscope and level 105. Since motion and orientation of the emitter and sensor may affect readings, the device should detect levelness and stillness within a certain predetermined tolerance before taking a glucose measurement. These additional detection hardware components of the device are operative connected to the processing unit and known generally in the art such as the accelerometer, gyroscope and level disclosed in U.S. Pat. No. 8,075,499 by Nathan et al., and U.S. Patent Application Pub. No. 20060212097 to Varadan et al.

The internal software and/or firmware of the device would include code having the ability to save and recall data, view at-a-glance glucose measurements in real-time (highs and lows), alarms for meals or snacks, easy-to-read recommendations, arrows showing trends of the user's blood glucose levels, scroll-though graphs for patterns of the user's blood glucose level, customizable predictive alerts for oncoming highs and lows by flashing icons or audible alarms (even if the device is set to a vibrate only mode), telecommunication updates, emergency related information, automated 911 calling, and the like. The device may also be connected to cell phones and could be activated by voice command, such as through Apple's Siri® or other voice recognition software. Additional features would include some standard features found in other watches or cell phones, such as time, day, date, a calendar, battery life, satellite location, weight, body temperature, climate, weather, atmospheric pressure, various languages the watch could display or understand, and control of brightness.

### Method

### Sampling Process and Algorithmic Processing

The device described above has the ability to accurately detect and measure glucose and other substances by using an algorithm that sorts, compares, and derives a measurement from samples. Preferable, the emitter unit 84 sends pulses to the sensing unit 86 at 30 terahertz pulses per second if the sensing array 68 is sufficiently still and dark.

In one embodiment of the method, the device only detects analytes if the following conditions are met: 1) the sensor unit 86 must read a light pollution at or near zero (i.e. below a certain threshold), 2) the orientation of the sensor unit 86 is level or perpendicular, or within a certain threshold angle (such as within five degrees or less of the horizontal or perpendicular plane of the device), and 3) the accelerometer detects motion below a certain latent threshold.

If the above conditions are met, the quantum cascade laser 60 emits a pulse at or approximately the resonant frequency of the analyte to be detected. In a preferred embodiment, the quantum cascade laser 60 emits a pulse at or approximately 1.4 terahertz, per the resonant frequency of glucose. (See “Far-infrared vibrational models of polycrystalline saccharides” by Upadhya et al, Vibrational Spectroscopy 35.1 (2004): 129-143, for resonant frequencies of various saccharides such as glucose, mannose, galactose, fructose, maltose, lactose, which could also be detected using the same device and methods).

As the terahertz waves pass through the tissue of the user, analytes absorb some energy from the terahertz waves but allows some energy to pass through the wrist 116, The terahertz waves that pass through the tissue excite the individual indium antimonide sensors 76 on the sensor array 74. Depending on the excitation level of the individual sensors 76, the composition of the sensor becomes conductive. The excitation jump of specific molecules known to be excited at a specific resonant frequency will cause each individual sensor 76 paired with the emitting cell 70 on the emitting array 68 to allow a certain amount of voltage through, which is communicated to the CPU 108 or other type of logic chip or system on a chip. The measuring of the voltages of each individual sensor 76 can then be synthesized into a graph (preferably 14×25 2d graph) of voltages, which shows their positions, and how close the resonant frequency of the substance being sought is. By measuring the voltage of individual sensors 76, the frequency energy received by a given sensor 76 from its paired emitter 70 can be deduced, and from this, the values of the concentration of the analytes can be deduced.

### Calibration

The user calibrates the device by holding the device perpendicular so that the device can detect the presence and positioning of wrist bones 104, tendons, etc., and can account for their respective attenuation tendencies. FIG. 5 illustrates the device wrapped around a user's wrist. Initial detection is performed by the quantum cascade laser 60 sweeping through a range of known body tissue frequencies to assess tissue makeup and construct a plethysmographic map of body structures. This map construction may take between three and five seconds. The onboard RAM 112 of the device stores a database of similar body topographies (along with attendant traditional baseline readings for glucose and/or other analytes to be detected). A cluster of topographies that are most similar to the user's will be saved from this initial plethysmographic scan. The database of similar body topographies (with attendant traditional baseline readings) as used in the previous phase will then be used for comparison of individual terahertz pulses to determine if any of the pulses are compromised on any given individual sensor 76. Sensors with the least obstructed, highest fidelity reception of the terahertz pulse, per the body topography map, are given priority, while sensors showing interference are given a lower priority due to low fidelity of the measured signal.

To establish a preliminary reading of the analyte, measurements of individual cells of the sensors showing the highest fidelity are added. Sensor cells not having the highest fidelity, in order of the algorithm's confidence in their usefulness, are compared to known instances of that same preliminary reading and its attendant outliers. A combined measurement using both the highest fidelity and lower fidelity sensor cell 76 readings are added, leading to either a higher or lower measurement of the analyte.

In other embodiments, the QCL may perform a broader and deeper sweep through frequencies associated with the analyte being sought. This allows the device to calculate the presence of a sought analyte from near misses, reflections, refractions, and other similar interference noise, in addition to the obvious resonant jumps in excitation. A control library of common yields from such sweeps and their attendant patterns will be stored in the onboard memory of the device will work in conjunction with the plethysmograph created at initial calibration.

### Alignment Offset Parsing Phase

Although the user will align the emitting array 68 with the sensor array 74 across their wrist or other body part, an exact alignment may not be possible. To mathematically pair each photo-conductive sensor 76 with the proper emitting sensor 70, the sensing unit 84 will determine the positioning of the emitting array 68 relative to the sensor array 74. This is accomplished by determining the x-position of the furthest edges of the sensor array 68 and measuring which photo-conductive sensors 76 are activated by beams from the QCL 60 (within a specified margin of error). Photo-conductive sensors 76 that are inactive for this sampling cycle and will store that cell count as a potential margin of error and compare subsequent sampling cycles to it until the watch is taken off. The positional offset of the emitter array 68 with the sensor array 70 will likely be offset by a different amount each time the user places the device on him or herself owing to differences in both the user's body and the variable sin exact positioning/angles/tightness of the band.

### Aggregation of the Sensor Readings

Sensors receiving the highest fidelity terahertz pulses are weighted in order of the algorithm's confidence and outlier readings from sensors having the least fidelity are compared to past distributions of outliers received from a set of measurements with a similar preliminary reading. This aggregation is performed because anatomical parts (e.g. a wrist) are not homogenous and the nature of determining topography with multiple terahertz pulses requires that several readings of terahertz pulses should be combined to form a reliable plethysmograph. Readings from the outliers are stored for the following step of the aggregation of signals. Certain outlier patterns will be given more weight, depending on the plethysmograph and how it compares to the outlying signals received in similar cases from the onboard database. Other outliers will be discarded from analysis as relegated to interference noise relative to the signal represented by the range of the composite. Based on the aggregation, the net reading will be adjusted higher or lower, depending on which weighting of the two readings is used and comparing the user to the stored cluster most similar to the user.

### Final Output

The net readings are averaged and compared to the device's library of known readings. The filtered outlier set is averaged and added to the average net readings. The result yields a compositional and volumetric analyte reading, which is extrapolated to account for the user's body volume/composition and saved in the memory of the device. The analyte measurement may be displayed on the screen of the device or transmitted to one or more than one other device such as device that a parent, guardian, physician, or emergency responder might have. The final output may be transmitted wirelessly via a transmitting module connected to the processing unit, logic chip, or system on a chip 108.

### Adaptability to Monitor Other Organic Molecules

Most organic molecules are structured in such a way that they resonate somewhere in the terahertz spectrum. A Quantum Cascade Laser can be tuned to any of the respective resonant frequencies of an organic molecule. The same algorithm used to calculate glucose may be used to determine the concentration of other analytes in blood using essentially the same algorithm described. Further embodiments of the method and devices include detecting multiple organic compounds simultaneously during the same reading. For example, glucose and ethanol would be a useful combination of analytes to be measured simultaneously.

What is claimed is:

1. A device for non-invasively measuring analytes in a biological being, such as, but not limited to blood glucose levels in a human, the device comprising:
a) a miniaturized quantum cascade laser (QCL) adapted to emit a plurality of terahertz radiation pulses;
b) an emitter unit operative connected to the QCL, the emitter unit comprising a fiber optic array comprising an array of field emission points;
c) a sensor unit comprising a photoconductive array adapted to receive the plurality of terahertz radiation pulses generated by the QCL, wherein the photo-conductive array has a plurality of individual photo-conductive sensors each comprising a positive terminal, a negative terminal, and a region of semi-conductive material sensitive to terahertz radiation between the positive and negative terminal;
d) a display unit adapted to display at least one measurement of an analyte measured by the device; and,
e) a processing unit comprising or operatively connected to programmable memory, a random access memory, the QCL, the sensor unit, and display unit, wherein the processing unit is configured to determine the concentration of an analyte;
wherein the emitter unit and the sensor unit are operatively connected to each other and designed to align substantially parallel with each other;
wherein the emitter unit and the sensor unit are designed to be placed on external surfaces of a biological being.

2. The device of claim 1, further comprising a tuning module operatively connected to the QCL, wherein the tuning module is capable of changing a frequency of the terahertz radiation pulses emitted by the QCL.

3. The device of claim 1, wherein the device is wearable by a person.

4. The device of claim 3, wherein the device is capable of being secured around a wrist of a person.

5. The device of claim 4, wherein the device further comprises:
a) a lower strap having a USB connector at a first end and a USB connector at a second end;
b) an upper strap;
c) a pocket connecting the upper strap and the lower strap, wherein the pocket is sized to fit the emitter unit, and wherein the pocket has a hole on its lower surface adapted to allow the array of field emissions points to be placed directly on a user's wrist; and,
d) an adjustable strap having a USB connector adapted to connect the lower strap to the upper strap, wherein the adjustable strap houses the sensing unit, and wherein the adjustable strap allows a user to align the array of field emission points with the photo-conductive array substantially parallel with each other.

6. The device of claim 5, wherein the upper strap comprises a plurality of protrusions along a length of the upper strap, and the adjustable strap comprises at least one hole,
wherein the plurality of protrusions are sized and shaped to securely fit within the at least one hole thereby allowing the user to adjust an overall length of the wrist watch by selecting one of the plurality of protrusions to fit within the at least one hole of the adjustable strap.

7. The device of claim 1, wherein the processing unit comprises a stored programmable memory, a random access memory, and the device is configured to measure and store a value a concentration of a blood component.

8. The device of claim 3, wherein the device is adapted to be inserted over a finger or adapted to be securely attached to an earlobe.

9. The device of claim 1, further comprising an accelerometer, a level, and a wireless transmitter, wherein the wireless transmitter is adapted to i) transmit a measurement of an analyte to a third party, and ii) transmit an alert signal.

10. The device of claim 9, wherein the alert signal is characterized as being a low blood sugar alert signal or a high blood sugar alert signal.

11. The device of claim 1, wherein the photo-conductive array comprises a plurality of individual indium antimonide photo-conductive sensors.

12. The device of claim 1 wherein the QCL emits terahertz radiation at a frequency of or about 1.4 terahertz through a wrist of a user, thereby allowing the device to measure blood glucose concentration of the user.

13. The device of claim 3, further comprising an gasket on a surface of the device, wherein the gasket designed to stabilize the device in a preset position when air or water is pumped within the gasket, and wherein the gasket is designed to reduce atmospheric radiation and visible light from contacting the photo-conductive array when air is pumped within the gasket.

14. A method of measuring a concentration of an analyte in a biological being, such as, but not limited to blood glucose concentration in a person, the method comprising the steps of:
generating electromagnetic waves in a terahertz range using a device comprising a miniaturized quantum cascade laser (QCL);
emitting electromagnetic waves in a terahertz range via a fiber optic array having plurality of field emission points arranged two-dimensionally;
transmitting electromagnetic waves in the terahertz range through a biological tissue;
measuring transmitted electromagnetic waves using a photo-conductive sensor array, wherein the photo-conductive sensor array comprises a plurality of individual photo-conductive sensors arranged two-dimensionally, and wherein the photo-conductive sensor array is positionally arranged parallel to the fiber optic array on opposite sides of the biological tissue; and,
calculating a value of an analyte from the transmitted waves by determining a frequency energy received by the photo-conductive sensor.

15. The method of claim 14, further comprising:
measuring a vertical and horizontal orientation of the device;
measuring a speed of the device;
measuring ambient light contacting the photo-conductive sensor; and,
emitting an electromagnetic wave in the terahertz range only in the event that i) the device is substantially horizontal and substantially vertical, ii) the device is substantially still, and iii) ambient light contacting the photo-conductive sensor is measured below a predetermined threshold.

16. The method of claim 14, wherein generating electromagnetic waves is characterized as generating electromagnetic waves between 0.3 terahertz and 3.0 terahertz.

17. The method of claim 16, wherein the analyte is glucose, and wherein generating electromagnetic waves is characterized as generating electromagnetic waves of or about 1.4 terahertz.

18. The method of claim 14, wherein emitting comprises emitting pulsed waves into the biological tissue.

19. The method of claim 15, wherein transmitting electromagnetic waves comprises transmitting electromagnetic waves from a top surface of a user's wrist to a bottom surface of a user's wrist.

20. The method of claim 13, further comprising the steps of,
calibrating the device by emitting a plurality pulses of electromagnetic waves in a terahertz range at a plurality of different frequencies;
generating a 2d graph of voltages measured at each of the plurality of individual photo-conducive sensors;
assessing tissue topography of user between the photo-conductive sensor of the device and the fiber optic array;
comparing tissue topography of a biological subject to a database of stored tissue topographies within the device;
measuring a fidelity of a received electromagnetic wave at each of the individual photo-conductive sensors;
weighing high fidelity measurements from individual photo-conductive signals higher than low fidelity measurements of individual photo-conductive signals in a calculation to determine a value of the analyte;
calculating a value of the analyte by comparing measurements of a user to a database of known measurements of the analyte; and,
displaying the value on the device.
